(19) United States
(12) Patent Application Publication
(10) Pub. No.: US 2009/0204187 A1
(43) Pub. Date: Aug. 13, 2009

(54) SOIL-BASED COMPOSITIONS AND METHODS FOR REMOVAL OF TOXINS FROM MAMMALS

(76) Inventor: Roy J. Mankovitz, Montecito, CA (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/369,826

(22) Filed: Feb. 12, 2009

Related U.S. Application Data
(60) Provisional application No. 61/027,998, filed on Feb. 12, 2008.

(51) Int. Cl. A61N 5/06; A61K 35/74; A61P 17/00; A61P 39/00

(52) U.S. Cl. 607/88; 424/93.4; 424/93.462; 424/93.46

(57) ABSTRACT

Humans and other mammals are continually exposed to toxins in the environment, toxins in ingested food and water, and toxins formed in the body through metabolism or breakdown of complex material. Such toxins impair health and contribute to disease in mammals but are difficult to avoid given the conditions of modern life. The present invention provides compositions for promoting removal of toxins from a subject which include an alkalizing agent, spore-forming bacteria, clay, at least one type of humic acid and/or at least one type of fulvic acid. Methods for use of compositions for promoting removal of toxins from a subject are described.

### REFERENCE TO RELATED APPLICATION

This application claims priority from U.S. Provisional Patent Application Ser. No. 61/027,998, filed Feb. 12, 2008, the entire content of which is incorporated herein by reference.

### FIELD OF THE INVENTION

The present invention relates generally to methods and compositions for promoting health in a subject. The present invention relates specifically to methods and compositions for promoting removal of toxins from a subject.

### BACKGROUND OF THE INVENTION

Humans and other mammals are continually exposed to toxins in the environment, toxins in ingested food and water, and toxins formed in the body through metabolism or breakdown of complex material. Such toxins impair health and contribute to disease in mammals but are difficult to avoid given the conditions of modern life.

### SUMMARY OF TEE INVENTION

Methods of promoting removal of toxins from a subject are provided according to embodiments of the present invention which include administering a therapeutically effective dose of a composition comprising an alkalizing agent, spore-forming bacteria, clay, humic acid and/or fulvic acid.

Alkalizing agents included can be one or more citric acid salts, one or more bicarbonate salts, one or more dried fruits, one or more fresh fruits or a combination of any two or more of these.

Spore-forming bacteria that can be included are illustratively B. subtilis, B. licheniformis, B. cereus, B. megaterium, B. clausii, B. coagulans, B. laterosporus or a combination of any of two or more of these. In preferred embodiments, spore-forming bacteria included in inventive compositions are B. coagulans, B. laterosporus or a combination B. coagulans and B. laterosporus.

Clay included in embodiments of inventive compositions is a natural clay, preferably a smectite clay. Optionally, the smectite clay is calcium bentonite, sodium bentonite or both calcium bentonite and sodium bentonite.

Methods of promoting removal of toxins from a subject are provided according to embodiments of the present invention which further include intentionally electrically grounding the subject for at least 1 hour-24 hours, inclusive, per day.

Methods of promoting removal of toxins from a subject are provided according to embodiments of the present invention which further include administering infrared radiation having a wavelength in the range of about 5 microns-20 microns, inclusive, to the subject for at least 1 hour-24 hours, inclusive, per day. In preferred embodiments, the administered infrared radiation has a wavelength of about 10 microns.

Compositions for promoting removal of toxins from a subject are provided according to embodiments of the present invention which include an alkalizing agent, spore-forming bacteria, clay, one or more humic acids and/or one or more fulvic acids.

Methods of inhibiting dandruff in a subject are provided according to embodiments of the present invention which include administering a therapeutically effective amount of a composition comprising isolated spore-forming bacteria and/or an isolated soil component. The isolated soil component is at least one type of humic acid and/or at least one type of fulvic acid in embodiments of inventive compositions and methods.

A pharmaceutically acceptable carrier can be included and optionally the pharmaceutically acceptable carrier includes clay.

Compositions for inhibiting dandruff in a subject are provided according to embodiments of the present invention which include isolated spore-forming bacteria and/or an isolated soil component. A pharmaceutically acceptable carrier can be included and optionally the pharmaceutically acceptable carrier includes clay.

### DETAILED DESCRIPTION OF THE INVENTION

Compositions and methods promoting removal of toxins from mammals are provided according to the present invention. Further advantageous effects of inventive compositions include stimulation of the immune system of an animal, nutritive value, and antimicrobial activity. Antimicrobial activity is direct, e.g. antibiotic producers, and/or indirect, e.g. inhibition of undesirable microbes by competitive exclusion.

Compositions for removal of toxins from mammals according to the present invention are “soil-based” and include three soil components and an alkalizing agent. Compositions of the present invention are believed to provide a synergistic detoxification effect, the components more effective in combination that the individual components alone.

The soil-based components are isolated spore-forming bacteria, isolated clay and isolated complex organic materials commonly found in soil, namely humic acids and/or fulvic acids.

The term “isolated” used herein refers to a component which is substantially separated or produced apart from substances with which the component naturally occurs. The term “isolated” does not implicate absolute purity of the component but is intended as a relative term. In preferred embodiments, the isolated component is at least 90% pure such that the component represents at least 90% of a preparation of the component used to make a composition of the present invention. In further preferred embodiments, the isolated component is at least 95% or greater pure such that the component represents at least 95% or greater of a preparation of the component used to make a composition of the present invention. Thus, for example, compositions according to embodiments of the present invention are prepared by combining: isolated spore-forming bacteria which is at least 90% pure, preferably at least 95% or greater pure, isolated clay which is at least 90% pure, preferably at least 95% or greater pure and isolated complex organic materials commonly found in soil, namely one or more humic acids and/or one or more fulvic acids, which are at least 90% pure, preferably at least 95% or greater pure.

Spore-forming bacteria included in compositions of the present invention include bacilli and clostridia which are not mammalian pathogens in dosage amounts used in the present invention. Such spore-forming bacteria are described in detail in standard references such as Bergey's Manual of Systematic Bacteriology, Springer-Verlag, 2nd Ed. 2005. Bacteria can be obtained from any of various sources for inclusion in an inventive composition, such as a commercial source, a repository, such as the American Type Culture Collection (ATCC), or by isolation from a natural source, such as soil. Methods for isolation and identification of spore-forming bacteria are known in the art.

Particular spore-forming bacteria included in embodiments of compositions are B. subtilis, B. licheniformis, B. cereus, B. megaterium, B. clausii, B. coagulans, and B. laterosporus.

A preferred embodiment of a composition of the present invention includes B. coagulans, and/or B. laterosporus.

Clay included in a composition of the present invention is preferably a natural clay. Preferred natural clays are smectites, particularly calcium bentonites and sodium bentonites illustratively including, and not limited to, Pascalite, Terramin and Redmond clay. Natural clays are obtained commercially or isolated from the natural environment.

Clay included in an inventive composition is preferably in the form of hydrated clay or dry powdered clay.

Humic acids and/or fulvic acids are included in compositions of the present invention. Humic acids and fulvic acids are a subset of humic substances, described in detail in references such as “Humus Chemistry: Genesis, Composition, Reactions”, F. J. Stevenson, John Wiley & Sons, New York, 2^{nd} Ed., 1994. Humic acids and fulvic acids are available commercially and can be isolated from natural sources such as soil and peat.

An alkalizing agent is included in compositions of the present invention for promoting a pH neutral or alkaline environment in the body of a mammal to which the composition is administered, as reflected by the value of urine pH. In particular, the soil-based components of an inventive composition, spore-forming bacteria, clay and humic and/or fulvic acids are believed to be most active to bind and retain toxins, and thereby promote removal of toxins from a mammalian body, in a neutral or alkaline environment.

Alkalizing agents illustratively include salts of citric acid such as calcium citrate, magnesium citrate, zinc citrate, selenium citrate, manganese citrate, chromium citrate, molybdenum citrate, potassium citrate, boron citrate and vanadium citrate. Alkalizing agents include mineral citrates such as potassium citrate, calcium citrate and magnesium citrate. Mixtures of citrates are included in particular compositions.

Bicarbonates are another alkalizing agent optionally included in inventive compositions. In particular, ammonia, calcium, potassium and/or sodium bicarbonate can be included.

Fruit is an alkalizing agent included in compositions of the present invention. Fruit is included in any form suitable for promoting an alkaline environment in a mammalian body, including but not limited to dried fruit and fresh fruit.

Methods of producing a composition for promoting removal of toxins from a subject are provided according to embodiments of the present invention which include combining an alkalizing agent, one or more types of isolated spore-forming bacteria, isolated clay, and at least one isolated type of humic acid and/or at least one isolated type of fulvic acid.

Amounts of the individual components in a composition of the present invention are each independently in the range of about 0.1-99% by weight of the total composition. One or more pharmaceutically acceptable carriers or excipients are optionally included in the composition.

In a particular embodiment, a composition of the present invention is administered contained in an ingestible capsule for oral ingestion. Capsule formulation is optionally designed to target release of the contained composition of the present invention to a particular target region, such as the stomach, small intestine and/or or large intestine.

Detailed information concerning customary excipients, equipment and processes for preparing dosage forms is found in Pharmaceutical Dosage Forms: Tablets, eds. H. A. Lieberman et al., New York: Marcel Dekker, Inc., 1989; and in L. V. Allen, Jr. et al., Ansel's Pharmaceutical Dosage Forms and Drug Delivery Systems, 8th Ed., Philadelphia, Pa.: Lippincott, Williams & Wilkins, 2004; A. R. Gennaro, Remington: The Science and Practice of Pharmacy, Lippincott Williams & Wilkins, 20th ed., 2003; and J. G. Hardman et al., Goodman & Gilman's The Pharmacological Basis of Therapeutics, McGraw-Hill Professional, 10th ed., 2001.

Methods of the present invention include administration of a composition including three soil components and an alkalizing agent substantially as described herein to a mammalian subject in order to promote removal of toxins from the subject.

Methods of promoting removal of toxins from a subject are provided according to embodiments of the present invention which include administering a therapeutically effective amount of a composition of the present invention.

The term “therapeutically effective amount” refers to an amount that elicits a desired therapeutic effect or provides a desired benefit. A preferred therapeutically effective amount is an amount which promotes removal of toxins from the subject. A further preferred therapeutically effective amount stimulates the immune system of the subject, provides nutritive value and provides antimicrobial activity. A further preferred therapeutically effective amount causes the subject to produce urine in a pH neutral or alkaline state. Therapeutically effective amounts and therapeutic effects of compositions and methods of the present invention can be assessed using any of various assays known to one of skill in the art.

Administration can include a single dose or multiple doses. Multiple doses are optionally administered each day and/or over the course of several days-weeks. In a further option, multiple doses are administered over a longer period, such as weeks-years, or indefinitely.

Exemplary non-limiting dosage ranges are described below.

Spore forming bacteria are administered in amounts of about 10⁴-10¹² colony forming units (CFU)/dose. Typically about 1 to 20 doses per day are administered.

Clay is administered in amounts of about 100 mg-1 gram/dose and about 1-60 grams per day are administered.

Humic acids and/or fulvic acids are administered in amounts of about 50 mg to 1 gram per dose. Typically, about 1 to 6 doses per day are administered.

An alkalizing agent is administered in an amount effective to maintain the urine produced by the subject in a pH neutral or alkaline state. Thus, in a subject treated with a composition of the present invention, the pH of urine can be monitored one or more times each day. Additional doses or a dosage form with more alkalizing agent is administered as necessary so that the subject produces urine in a pH neutral or alkaline state.

Any effective amount of an alkalizing agent can be included in a composition of the present invention.

For example, a citrate-based alkalizing agent can be administered in amounts of about 0.1 g-10 g/dose. Typically about 1 to 10 doses per day are administered.

A dose of a specific citrate-based alkalizing agent includes 300 mg calcium citrate, 270 mg magnesium citrate, 21 mg zinc citrate, 150 mcg selenium citrate, 6 mg manganese citrate, 200 mcg chromium citrate, 90 mcg molybdenum citrate, 90 mg potassium citrate, 3 mg boron citrate and 90 mcg, vanadium citrate.

In a further example, 4-8 g of potassium citrate is administered per day in a single dose or divided among multiple doses.

Exemplary doses of bicarbonate include but are not limited to 1-300 mEq. Typically about 1 to 10 doses per day are administered.

Fruit is also optionally included in compositions and administered in amounts effective to maintain the urine produced by the subject in a pH neutral or alkaline state. Many fruits contain citrates and are administered in amounts sufficient to deliver a citrate-based alkalizing agent in amounts of about 0.1 g-10 g/dose. Typically about 1 to 10 doses per day are administered.

Compositions for promoting removal of toxins from a subject are formed by combining active ingredients which can each individually be in solid, liquid or semi-liquid form.

Methods of promoting removal of toxins from a subject are provided according to embodiments of the present invention which further include electrically grounding the subject for at least 1-24 hours/day.

Until relatively recently, human beings walked barefoot and slept directly on the earth, which is electrically neutral. In the last few decades, however, the use of shoes having non-conductive soles, synthetic carpeting and other floor materials have insulated people and animals from the earth. Studies have shown that earth grounding is an important source of free radicals which function as natural antioxidants. It is believed that the build-up of electrical charge in a human body may have detrimental side-effects. Research has indicated that stored charge may interfere with normal cellular communications and the self-regulating and self-healing mechanisms of the body, which in turn may create stress and lead to certain maladies. In particular, electrostatic charge build-up may adversely affect the nervous system, causing muscle stiffness and back pain. In this physiologically stressed state, blood pressure rises, heart rate increases, and the digestive process slows. It is believed that the unnatural presence of electrostatic charges on the body may also have a correlation with certain diseases, at least partly because electrical isolation interferes with normal immune system function and increases inflammation.

Electrical grounding of a subject is achieved, for instance, using an apparatus that includes an electrically conductive element having a first end connected to an electrical ground and a second end in contact with the subject directly or indirectly, such as by connection to an electrically conductive element of an object connected to the electrical ground. Such objects are preferably articles that the subject contacts frequently or for extended periods of time such that the subject is intentionally electrically grounded for at least 1-24 hours/day. Such objects illustratively include an electrically conductive portion of a computer mouse or keyboard, an electrically conductive portion of an article of furniture such as a chair or bed, an electrically conductive portion of a housing for an animal or an electrically conductive portion of a member within the housing such as an electrically conductive perch, an electrically conductive food or water bowl, an electrically conductive sleeping pad, or an electrically conductive food or water bowl.

Electrical grounding apparatus for use in methods according to embodiments of the present invention can include existing devices modified to electrically ground a user and/or use of methods and/or devices disclosed in U.S. Pat. Nos. 6,683,779; 7,212,392 and U.S. Application Publication Nos. 2006/0285266 and 20080022941.

Methods of promoting removal of toxins from a subject are provided according to embodiments of the present invention which further include exposing the subject to Far Infrared radiation for at least 1-24 hours/day.

Far infrared radiation, having a wavelength of about 5-20 microns, inclusive, preferably having a wavelength of about 10 microns, is administered to heat at least portions of the body of a subject according to embodiments of inventive methods. Methods of the present invention preferably include administration of far infrared radiation to a subject using a far infrared heater or heating pad. The infrared heater produces radiant energy, which is the same as the heat from the sun, only without the harmful ultraviolet rays. The radiation penetrates the body and heats through a process called conversion, instead of heating the air around the user.

It is believed that administered far infrared heat penetrates more than 1.5 inches (40 mm) into the body. The argument is based on the idea that the wavelengths of far infrared waves are typically between 5.8 and 1000 micrometers. This is supposed to correspond to the vibration of the water molecule at 9.4 micrometers. Because these vibrations are similar, the infrared rays help release toxins from fat cells and other cells, and the toxins can be released through sweating or other mechanisms of bodily elimination. Far infrared radiation is directed to any area for which a therapeutic effect is desired, illustratively including whole body, kidneys, sinuses and intestines.

A subject treated according to a method of the present invention can be any mammal, including human and non-human primates, cats, dogs, horses, sheep, goats, cattle, pigs, rabbits, and rodents. In preferred embodiments, the subject is human.

Compositions and methods are provided according to the present invention for inhibiting Melassezia fungal infection of the scalp which causes dandruff in an affected subject.

Malassezia fungi require exogenously supplied lipids since these organisms are incapable of producing metabolically required fatty acids. As a consequence, Malassezia fungi are frequently found associated with sebaceous glands on the scalp, causing dandruff.

Methods of treating a subject to inhibit dandruff include administering a composition including a therapeutically effective amount of isolated spore-forming bacteria and/or an isolated soil component which is at least one type of humic acid and/or at least one type of fulvic acid.

One or more pharmaceutically acceptable carriers or excipients are optionally included in the composition for inhibiting dandruff, with the proviso that no lipids or fatty acids are included in the carrier(s) or excipient(s) since these substances enhance Malassezia fungi growth. Conventional pharmaceutically acceptable carriers or excipients typically used in formulations for application to the scalp or skin of a subject can be used with the proviso that no lipids or fatty acids are included in the carrier(s) or excipient(s).

In embodiments of the present invention, clay is a pharmaceutically acceptable carrier included in a composition for inhibiting dandruff including a therapeutically effective amount of isolated spore-forming bacteria and/or an isolated soil component which is at least one type of humic acid and/or at least one type of fulvic acid.

The spore-forming bacteria and/or the soil component are each individually present in a composition for treating a subject to inhibit dandruff in an amount in the range of 1% weight/total weight of the composition-99% or greater weight/total weight of the composition or up to saturation of the carrier.

The term “pharmaceutically acceptable carrier” refers to an organic, inorganic, liquid, or solid, natural or synthetic substance suitable for administration to a subject which is combined with an active ingredient to facilitate administration and which is compatible with the active ingredient(s) such that the carrier does not substantially impair the desired therapeutic effect of the active ingredient(s).

The following references contain information relevant to the present application and are hereby incorporated by reference in their entirety for all purposes.

### REFERENCES

Christi, I., et al., Relating ion binding by fulvic and humic acids to chemical composition and molecular size. 2. Metal binding. Environ. Sci. Technol, 2001. 35(12): p. 2512-2517.

Liu, A. and R. D. Gonzalez, Adsorption/Desorption in a System Consisting of Humic Acid, Heavy Metals, and Clay Minerals, Journal of Colloid And Interface Science, 1999. 218(1): p. 225-232.

Mullen, M. D., et al., Bacterial sorption of heavy metals. Appl Environ Microbiol, 1989. 55(12): p. 3143-3149.

Beveridge, T. J. and R. G. E. Murray, Uptake and retention of metals by cell walls of Bacillus subtilis. J. Bacteriol, 1976. 127(3): p. 1502-1518.

Fowle, D. A. and J. B. Fein, Experimental measurements of the reversibility of metal-bacteria adsorption reactions. Chemical Geology, 2000. 168(1-2): p. 27-36.

Proudfoot, A. T., E. P. Krenzelok, and J. A. Vale, Position Paper on Urine Alkalinization. Clinical Toxicology, 2005. 42(1): p. 1-26.

Minich, D. M. and J. S. Bland, Acid-Alkaline Balance: Role In Chronic Disease And Detoxification. Physiology. 6(11): p. 12.

Diamond, J., Eat Dirt, in Discover Magazine. 1998. p. pp 70-76.

Dextreit, R., Our Earth, Our Cure. 1974: Swan House.

A˜, P., Living Clay. 2006: Perry Productions.

Abehsera, M., The Healing Power Of Clay. 2001: Citadel.

Callahan, G. N., Eating dirt. Emerg Infect Dis, 2003. 9(8): p. 1016-1021.

Graham, C., The Clay Disciples. 2006.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art.

1. A method of promoting removal of toxins from a subject, comprising:
administering a therapeutically effective dose of a composition comprising an alkalizing agent, spore-forming bacteria, clay, humic acid and/or fulvic acid.

2. The method of claim 1, wherein the alkalizing agent is selected from the group consisting of: a citric acid salt, a bicarbonate salt, dried fruit, fresh fruit and a combination of any two or more of these.

3. The method of claim 2, wherein the citric acid salt is selected from the group consisting of: calcium citrate, magnesium citrate, zinc citrate, selenium citrate, manganese citrate, chromium citrate, molybdenum citrate, potassium citrate, boron citrate and vanadium citrate and a combination of any of two or more of these.

4. The method of claim 2, wherein the bicarbonate salt is selected from the group consisting of: ammonia bicarbonate, calcium bicarbonate, potassium bicarbonate, sodium bicarbonate and a combination of any of two or more of these.

5. The method of claim 1, wherein the spore-forming bacteria are selected from the group consisting of: B. subtilis, B. licheniformis, B. cereus, B. megaterium, B. clausii, B. coagulans, B. laterosporus and a combination of any of two or more of these.

6. The method of claim 1, wherein the spore-forming bacteria are selected from the group consisting of: B. coagulans, B. laterosporus and a mixture B. coagulans and B. laterosporus.

7. The method of claim 1, wherein the clay is a natural clay.

8. The method of claim 7, wherein the natural clay is a smectite clay.

9. The method of claim 8, wherein the smectite clay is selected from the group consisting of: calcium bentonite, sodium bentonite and a mixture of calcium bentonite and sodium bentonite.

10. The method of claim 1, further comprising intentionally electrically grounding the subject for at least 1 hour-24 hours, inclusive, per day.

11. The method of claim 1, further comprising administering infrared radiation having a wavelength in the range of about 5 microns-20 microns, inclusive, to the subject for at least 1 hour-24 hours, inclusive, per day.

12. The method of claim 1, further comprising administering infrared radiation having a wavelength of about 10 microns to the subject for at least 1 hour-24 hours, inclusive, per day.

13. A composition for promoting removal of toxins from a subject, comprising:
an alkalizing agent, spore-forming bacteria, clay, humic acid and/or fulvic acid.

14. The composition of claim 13, wherein the alkalizing agent is selected from the group consisting of: a citric acid salt, a bicarbonate salt, dried fruit, fresh fruit and a combination of any two or more of these.

15. The composition of claim 13, wherein the spore-forming bacteria are selected from the group consisting of: B. subtilis, B. licheniformis, B. cereus, B. megaterium, B. clausii, B. coagulans, B. laterosporus and a combination of any of two or more of these.

16. The composition of claim 13, wherein the spore-forming bacteria are selected from the group consisting of: B. coagulans, B. laterosporus and a mixture B. coagulans and B. laterosporus.

17. The composition of claim 13, wherein the clay is a natural clay.

18. The composition of claim 17, wherein the natural clay is a smectite clay.

19. The composition of claim 18, wherein the smectite clay is selected from the group consisting of: calcium bentonite, sodium bentonite and a mixture of calcium bentonite and sodium bentonite.

20. A method of producing a composition for promoting removal of toxins from a subject, comprising:
combining an alkalizing agent, isolated spore-forming bacteria, isolated clay, and at least one isolated type of humic acid and/or at least one isolated type of fulvic acid.

21. A method of inhibiting dandruff in a subject, comprising:
administering a therapeutically effective amount of a composition comprising isolated spore-forming bacteria and/or an isolated soil component.

22. The method of claim 21, wherein the isolated soil component is at least one type of humic acid and/or at least one type of fulvic acid.

23. The method of claim 21, wherein the composition further comprises a pharmaceutically acceptable carrier, with the proviso that no lipids or fatty acids are included in the carrier(s) or excipient(s).

24. The method of claim 23, wherein the pharmaceutically acceptable carrier is clay.

25. A composition for inhibiting dandruff in a subject, comprising isolated spore-forming bacteria and/or an isolated soil component.

26. The composition of claim 25, wherein the composition comprises clay.

27. The composition of claim 25, wherein the composition comprises a pharmaceutically acceptable carrier, with the proviso that no lipids or fatty acids are included in the carrier(s) or excipient(s).
